(19) United States
(12) Patent Application Publication
(10) Pub. No.: US 2024/0141272 A1
(43) Pub. Date: May 2, 2024

(54) AEROSOL-COMPATIBLE CELL CULTURE EXPOSURE SYSTEMS

(71) Applicant: United States Government, as represented by the Administrator of the U.S. EPA, Washington, DC (US)

(72) Inventors: Jessica R. MURRAY, Durham, NC (US); Jason Paul WEINSTEIN, Holly Springs, NC (US); Quentin Todd KRANTZ, Chapel Hill, NC (US); Mark HIGUCHI, Chapel Hill, NC (US)

(21) Appl. No.: 17/977,114

(22) Filed: Oct. 31, 2022

(51) Int. Cl. C12M 1/00; C12M 1/32

(52) U.S. Cl. CPC C12M 23/40; C12M 23/12; C12M 23/38; C12M 29/06

(57) ABSTRACT

A unique aerosol-compatible cell culture exposure system is described that allow serial dilution and delivery of aerosols, reactive gases, and VOCs at air-liquid interface (ALI) under physiologically relevant conditions. Generally, the cell culture system comprise one or more dilution manifolds, one or more flow splitters, and a sealed exposure chamber that houses a commercially-available cell culture plate with corresponding transwells.

### FIELD

The present subject matter relates to a cell culture exposure system and related methods that enable high-throughput air-liquid interface exposures to multiple doses of particles or volatile compounds while maintaining appropriate cell culture conditions.

### BACKGROUND

Various prior art exposure systems were evaluated in their ability to achieve serial dilution and delivery of volatile organic compounds (VOCs), ultrafine particles/nanoparticles, i.e., typically less than 100 nm, or fine particles, i.e., typically less than 2.5 μm, at an air-liquid interface (ALI) and their ability to maintain physiologically relevant conditions, for example a temperature of 37° C., and relative humidity (RH) of greater than 80%.

Two systems in widespread use are commercially available under the designations CULTEX and VITROCELL. These systems offer dynamic exposure conditions for volatile compounds, such as cigarette smoke, e-cigarette vapor, and nanoparticles, but cannot be used reliably for fine particles, i.e., defined as an aerosol aerodynamic diameter less than 2.5 μm. An exception to this is the VITROCELL CLOUD system which includes an integrated nebulizer and promotes high deposition efficiency for particles via sedimentation within a confined, static chamber. An alternative nebulizer design by Harvard College is also available for larger droplets which are directly applied to a cell surface.

When the above noted exposure apparatuses are used in aerosol toxicity studies, there is often no clear definition of the aerodynamic particle size. In addition, dosimetry metrics are rarely reported which makes it difficult to assess reliability and repeatability of a system under review. As far as is known, all known systems are inadequate to satisfy the requirements of a higher throughput exposure system which can produce up to six doses of VOCs, ultrafine particles, i.e., typically less than 0.1 μm, or fine particles, i.e., typically less than 2.5 μm, with four technical replicates per dose within a standard 24-well cell culture plate to meet toxicity screening demands. In many biological or toxicological applications, such exposure systems must also maintain appropriate cell culture conditions, which are typically a temperature of 37° C., and a relative humidity of greater than 80%. Vehicle controls must also demonstrate minimal cytotoxicity compared to incubator controls.

### SUMMARY

The difficulties and drawbacks associated with previous approaches are addressed in the present subject matter as follows.

In one aspect, the present subject matter provides a cell culture exposure system which comprises of one or more dilution manifolds including at least one inlet for receiving fluid flow. The system also comprises at least one flow splitter. And, the system comprises an exposure chamber configured to receive a cell culture plate which contains permeable cell culture inserts which maintain cells at the air-liquid interface. Flow splitters are positioned between the outlets of the dilution manifold and the inlets to the exposure chamber to provide flow communication and ensure uniform deposition of delivered test agent across all downstream technical replicates.

In another aspect, the present subject matter provides a cell culture exposure system comprising a dilution manifold having at least one inlet for receiving fluid flow. The system also comprises an assembly of flow splitters. And, the system comprises an exposure chamber configured to receive a cell culture plate which contains permeable cell culture inserts in each well. The assembly of flow splitters is disposed between the dilution manifold and the exposure chamber and provides a flow communication therebetween. The system is configured such that each well of the plurality of wells receives its own independent flow of the fluid flow downstream of the assembly of flow splitters.

In still another aspect, the present subject matter provides a method for exposing a cell culture plate to a test fluid flow. The method comprises providing a cell culture exposure system including at least one dilution manifold including at least one inlet for receiving a fluid flow, at least one flow splitter, and an exposure chamber configured to receive a cell culture plate. The method also comprises providing a cell culture plate having at least one well. The method further comprises positioning cell culture material in the at least one well. The method additionally comprises placing the cell culture plate with cell culture material positioned therein, in the exposure chamber of the cell culture exposure system. And, the method further comprises directing a fluid flow into the at least one inlet of the at least one dilution manifold such that the fluid flow passes through the at least one well and the cell culture material positioned therein.

As will be realized, the subject matter described herein is capable of other and different embodiments and its several details are capable of modifications in various respects, all without departing from the claimed subject matter. Accordingly, the drawings and description are to be regarded as illustrative and not restrictive.

### BRIEF DESCRIPTION OF THE DRAWINGS

FIG. 1A is a schematic perspective view of an embodiment of a cell culture exposure system using an aerosol flow splitter in accordance with the present subject matter.

FIG. 1B is a schematic side elevational view of the cell culture exposure system illustrated in FIG. 1A.

FIG. 1C is a schematic perspective view of another embodiment of a cell culture exposure system using a VOC flow splitter in accordance with the present subject matter.

FIG. 2A is a schematic view of an inlet cap used in an embodiment of a dilution manifold of the cell culture exposure system of the present subject matter.

FIG. 2B is a schematic view of a first inner diluter used in an embodiment of a dilution manifold in the cell culture exposure system.

FIG. 2C is a schematic view of an exhaust dilutor used in an embodiment of a dilution manifold of the cell culture exposure system.

FIG. 3A is a schematic partially exploded view of an aerosol flow splitter of the cell culture exposure system.

FIG. 3B is a schematic perspective view of the aerosol flow splitter depicted in FIG. 3A.

FIG. 4A is a schematic partially exploded view of a VOC flow splitter of the cell culture exposure system.

FIG. 4B is a schematic perspective view of the VOC flow splitter shown in FIG. 4A.

FIG. 4C is a schematic side view of the VOC flow splitter illustrated in FIG. 4A.

FIG. 5A is a schematic partially exploded view of an exposure chamber of the cell culture exposure system of the present subject matter.

FIG. 5B is a schematic side elevational view of the exposure chamber illustrated in FIG. 5A.

FIGS. 6A-6B are schematic views of a base used in the exposure chamber of the cell culture exposure system.

FIGS. 6C-6D are schematic views of an annular plate used in the exposure chamber of the cell culture exposure system.

FIGS. 6E-6H are schematic views of a plenum plate used in the exposure chamber of the cell culture exposure system.

### DETAILED DESCRIPTION OF THE EMBODIMENTS

Recently, in vitro airway models grown at the air-liquid interface (ALI) have been used for inhalation toxicity screening. These advanced ALI cell cultures allow direct cell-toxicant interaction to mimic realistic inhalation exposures. In addition, these cultures are uniquely suited to handle methodologically challenging chemicals (MCCs) which include volatile and water-/DMSO-insoluble compounds. However, specialized ALI exposure systems must meet the following specifications to allow regulatory agencies to adopt and validate in vitro assays:

The system must maintain physiological temperature (T) and relative humidity (RH) under dynamic airflow conditions for several exposure types, such as aerosols, volatile compounds, and reactive gases. Vehicle controls, which are typically cells exposed to clean air without test articles, must demonstrate minimal cytotoxicity compared to incubator controls.

The system must also provide serial dilution of a test article with technical replicates to meet higher throughput screening demands and characterize dose-response relationships within a single exposure.

The system must also exhibit flexibility to accommodate inhalable test articles with different physicochemical properties and size metrics, for example, volatile organic compounds (VOCs), ultrafine particles/nanoparticles, i.e., typically less than 100 nm, and fine particles, i.e., typically less than 2.5 μm.

The system must further provide high repeatability. That is, the system should exhibit low standard deviations for particle deposition, which typically are less than 20% between technical replicates and inter-day repeat exposures.

To meet these demands, and in accordance with the present subject matter, a unique aerosol-compatible cell culture exposure system is provided that allow serial dilution and delivery or flow of a wide array of test fluids such as but not limited to aerosols, reactive gases, and VOCs at ALI under physiologically relevant conditions (T=37° C., RH>80%). The present subject matter system meets higher throughout demands for toxicity screening by producing six doses in a single exposure with four technical replicates per dose. Standard deviations of particle deposition between technical replicates are usually less than 10%. Another key feature of the present subject matter system is the direct utilization of standard 24-well cell culture plates with 6.5 mm permeable ALI inserts. This increases throughput, decreases sample handling time, and allows laboratories and evaluation facilities to use commercially available cell culture materials and equipment. Furthermore, the present subject matter system is primarily configured for 3D human airway models grown at ALI, for example, nasal epithelial cells, bronchial epithelial cells, alveolar epithelial cells. However, it is understood that the present systems are also compatible with any cell type grown at ALI, keratinocytes for dermal toxicity studies, or mutagenesis assays, for example an Ames assay.

It is contemplated that the present subject matter systems will be used by various regulatory agencies and other concerns to utilize new approach methods (NAMs) for inhalation toxicity screening in support of the Toxic Substances Control Act (TSCA) and Federal Insecticide, Fungicide, and Rodenticide Act (FIFRA).

Current risk assessment strategies are trending toward the reduction and refinement of the use of animal models for toxicity screening and the development and validation of alternative test methods. In vitro NAMs are now being deployed to meet the demands of risk assessment testing in a time- and cost-effective manner. However, appropriate commercially available ALI exposure equipment to support this transition is lacking. The US EPA Office of Chemical Safety and Pollution Prevention (OCSPP), Office of Pesticide Programs (OPP), and Office of Pollution Prevention and Toxics (OPPT) have requested in vitro inhalation data for volatile and non-volatile, water-/DMSO-insoluble compounds that have been nominated by TSCA for further toxicological evaluation. These regulatory partners have a key interest in developing improved in vitro inhalation assays to reduce the need for 90-day animal inhalation studies. This necessitates a specialized ALI exposure system that maintains physiologically relevant conditions, can accomplish serial dosing to achieve benchmark dose modeling of toxicological endpoint assays, such as viability, cytotoxicity, transcriptomics, etc., is flexible to accommodate a diverse range of VOCs and particles, and is repeatable to accomplish replicate studies and repeat-dosing studies.

Repeatable particle delivery is especially difficult because deposition mechanisms, for example, impaction, sedimentation, diffusion, and electrostatic precipitation, vary by particle size and physicochemical properties. This is an important consideration for risk assessment and in vitro study design due to the anatomy of the human respiratory tract and the distinct particle size ranges that penetrate to each region as follows.

Inhalable fraction particle sizes, for example less than 10 μm, generally include the mass fraction of total airborne particles which are inhaled through the nose and mouth. Most large particles from 5-10 μm usually deposit on the upper airways and are successfully cleared by macrophages. The primary deposition mechanism for such particle sizes is impaction. An applicable cell model is Nasal epithelial cells. Generally, the regulatory need is low to moderate. Thoracic fraction particle sizes, for example less than 3 μm in adults, and less than 5 μm in children, are generally the mass fraction of inhaled particles penetrating beyond the larynx and reaching the tracheobronchial region. Particles in the range of 1-2.5 μm are delivered to the terminal bronchiole which is the site of greatest accumulation and tissue destruction. The primary deposition mechanism for these particle sizes is sedimentation. An applicable cell model is Bronchial epithelial cells. Typically, the regulatory need is high. Respirable fraction, for example less than 1 μm of particle sizes, are the mass fraction of inhaled particles that gain access to the gas-exchange region, also referred to the alveolar or pulmonary region. Ultrafine particles and nanoparticles, for example less than 100 nm, deposit in the gas-exchange region. The primary deposition mechanism for such particle sizes is diffusion. An applicable cell model is Alveolar Type I and II epithelial cells. Generally, the regulatory need is high.

Given the wide range of real-world particle populations and deposition mechanisms in the human respiratory tract, an ALI cell culture exposure system must be able to deliver thoracic and respirable particle size ranges to meet risk assessment demands. Both particle size ranges are associated with high toxicity, but it is important to note that these particle groups are subject to different transport physics and deposition mechanisms. Current commercially available ALI exposure systems, for example the VITROCELL system, show strong performance metrics for nanoparticles and volatile compounds. However, the present subject matter systems are unique in their ability to also transport larger thoracic fraction particles, for example less than 2.5 μm, in a repeatable and high throughput manner while maintaining physiologically relevant conditions of relative humidity and temperature. As far as is known, CULTEX RFS is the only other commercially available system with a proven ability to transport inhalable and thoracic fractions of particles with low standard deviations. However, such system exhibits low throughput, for example, n=3, and no serial dilution, and fails to provide physiological relative humidity conditions which leads to high cytotoxicity of vehicle or sham control cells. The present subject matter systems can be broadly used by industry groups, contract research organizations (CROs), and regulatory agencies for chemical screening to aid in regulatory decision making.

The present subject matter provides a unique aerosol-compatible cell culture exposure system that allow serial dilution and delivery of aerosols, reactive gases, and VOCs at ALI under physiologically relevant conditions, for example a temperature of 37° C., and a relative humidity of greater than 80%, to accomplish higher throughput toxicity screening and the incorporation of in vitro NAMs in inhalation risk assessment. Flow parameters can be tuned or selectively adjusted to dilute and deliver VOCs via diffusion with scalar mixing principles, or operational flows can be reduced or otherwise varied to deliver repeatable particle doses via sedimentation and thermophoresis. Vehicle controls demonstrate minimal changes in viability, measured by ATP formation, cytotoxity, measured by lactate dehydrogenase (LDH), and barrier integrity, for example, trans-epithelial electrical resistance (TEER), when compared to incubator controls which is important for toxicological screening of test articles at the ALI.

The present subject matter also provides a modular ALI cell culture exposure system that provides higher throughput toxicological screening of inhaled toxicants by achieving one or more of the criteria described herein.

The systems of the present subject matter comprise modular, leak-proof components which allow the systems to be easily configured for VOCs or aerosols. Generally, the systems comprise at least one dilution manifold having an inlet for receiving a fluid, at least one flow splitter, and an exposure chamber configured to receive a cell culture plate. All of these components are described in greater detail herein.

A wide array of dilution manifolds can be used in the systems of the present subject matter. The dilution manifolds include one or more inner diluters, one or more exhaust diluters, and one or more inlet caps. In certain versions, the dilution manifold may also include a U-turn component. The U-turn component is disposed between two inner diluters and provides flow communication therebetween. The term “flow communication” is used herein and refers to a configuration in which flow of air, gas, or other fluid can readily occur between adjacent and typically adjoining components. Typically, such flow communication occurs through one or more passages defined in the components. As will be understood, the term is not limited to flow of air, but rather includes a wide array of flowable fluids, mists, aerosols, entrained particulates, gas-like slurries, and related collections of matter.

A variety of flow splitters may be used in the systems of the present subject matter. In one version, a VOC-specific flow splitter, herein referred to as a VOC flow splitter, includes a sampling port for real-time monitoring with gas chromatography (GC)-coupled detection methods, for example. In another version, an aerosol-specific flow splitter, herein referred to as an aerosol flow splitter, includes a static mixer to align particle flow paths for uniform deposition. These components are described in greater detail herein.

A combination of a dilution manifold and a flow splitter allows serial dilution and delivery of inhaled toxicants, for example, six (6) doses with four (4) technical replicates, which allows multiplexing endpoint assays.

In many embodiments, the exposure chamber accommodates standard cell culture plates with 6.5 mm ALI inserts and is fully compatible with VOCs or aerosols. In many embodiments, the exposure chamber is also equipped with heating element(s) to enhance deposition via thermophoresis while maintaining ideal cell culture conditions.

The system of the present subject matter can be utilized with different flow parameters to transport VOCs and/or aerosols and target different dose ranges. Each component of the present subject matter systems is configured for easy assembly, operation, and cleaning.

The operation of the system of the present subject matter provides serial dilution of a wide range of test articles, for example, VOCs, reactive gases, and particles, and delivers a plurality of doses with at least four technical replicates per dose to cells cultured at ALI in a standard 24-well plate outfitted with 6.5 mm inserts. In particular applications, the exposure system can be configured to deliver six doses with four technical replicates per dose. Typically, the exposure chamber is sealed and configured to maintain physiological relative humidity and temperature throughout dynamic cell exposure conditions. The dilution manifold is configured to balance inertial and gravitational forces that influence particle movement to minimize losses of fine particles, typically, less than 2.5 μm. A four-way flow splitter coupled with static mixers ensures uniform particle deposition. Technical replicates of particle deposition within a single dose achieve a standard deviation of less than 10%, indicating the repeatability and reliability of the present subject matter systems. A simple substitution of the flow splitter module and alignment of the dilution manifolds enables a system of the present subject matter to deliver volatile compounds and reactive gases. The present subject matter provides a flexible and adaptable exposure system that meet higher throughput demands and risk assessment needs.

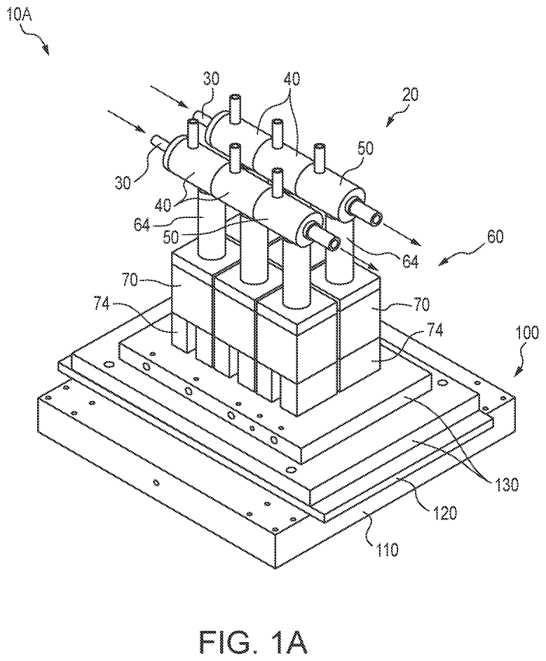

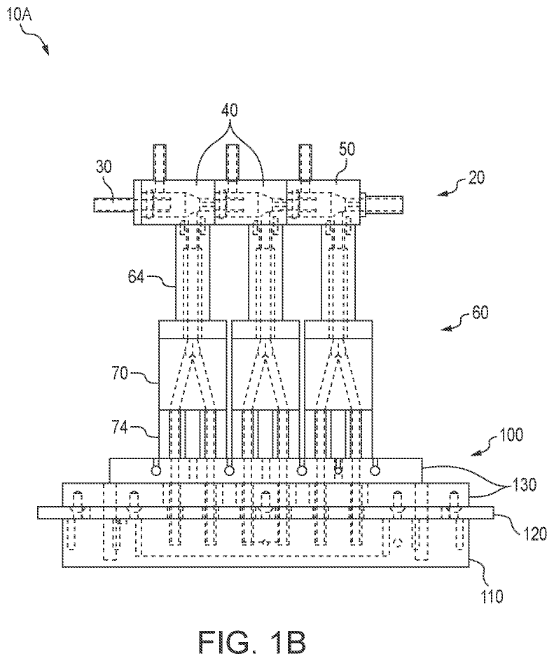

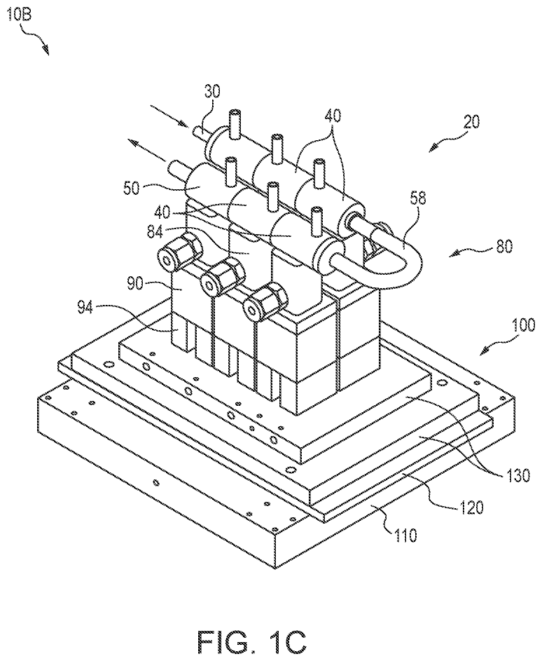

Referring to FIGS. 1A-1B, an embodiment of a cell culture exposure system in accordance with the present subject matter is shown. This system produces six (6) doses of particles with a dilution manifold that includes two aerosol inlets and two exhaust outlets. In this particular embodiment, the dilution manifold is a split dilution manifold having two sets of diluters arranged in parallel. Specifically, a cell culture exposure system 10A is illustrated comprising a dilution manifold 20, an assembly of aerosol flow splitters 60, and an exposure chamber 100. The assembly of aerosol flow splitters 60 is disposed between the dilution manifold 20 and the exposure chamber 100 and provides flow communication therebetween. The assembly of aerosol flow splitters 60 includes at least one and typically a plurality of aerosol flow splitters 60, described in greater detail herein.

In another embodiment shown in FIG. 1C, a cell culture exposure system comprises a dilution manifold, an assembly of VOC flow splitters, and an exposure chamber that is configured to fittingly engage a standard 24-well cell culture plate. The system is configured for VOC delivery by connecting two sets of diluters so they are in flow communication with each other, for example by connecting with a U-turn component and with a particular assembly of VOC flow splitters which includes sampling ports for real-time detection of volatile compounds as shown in FIG. 1C. Specifically, a cell culture exposure system 10C is depicted comprising a dilution manifold 20, an assembly of VOC flow splitters 80, and an exposure chamber 100. The assembly of VOC flow splitters 80 is disposed between the dilution manifold 20 and the exposure chamber 100 and provides flow communication therebetween. The assembly of VOC flow splitters 80 includes at least one and typically a plurality of VOC flow splitters 80, described in greater detail herein.

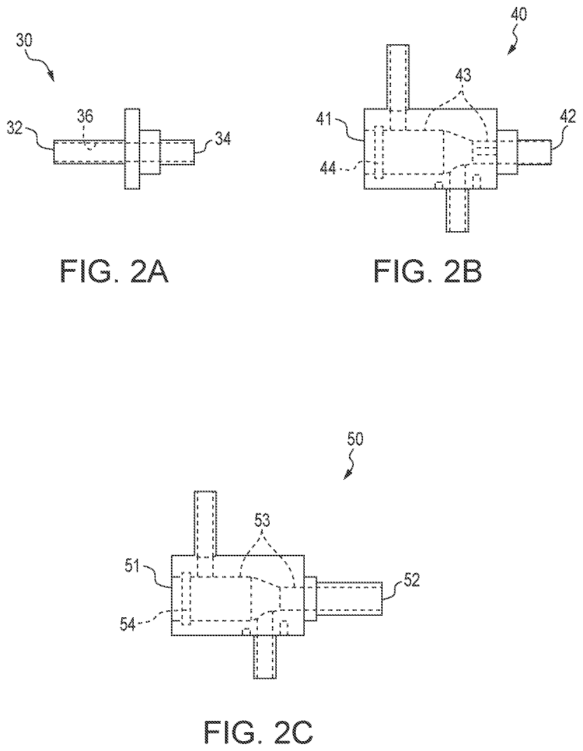

The dilution manifold 20 typically includes at least one inlet cap 30, at least one inner dilutor 40, and at least one exhaust dilutor 50 as illustrated in FIGS. 2A-2C. O-rings are used to achieve a tight seal between adjoining component surfaces. And the size of inlets and outlets correspond with tubing fittings such as those commercially available from Swagelok for ease of use. Internal geometry is configured to minimize particle loss while achieving serial dilution of the test article. The geometry and corresponding flow parameters can be optimized with humidified air, for example greater than 80% relative humidity, to ensure physiological conditions are maintained throughout the course of an exposure. Specifically, FIG. 2A illustrates an inlet cap 30. The inlet cap 30 defines an inlet port 32, and outlet port 34, and a passageway 36 extending therebetween. FIG. 2B illustrates an inner diluter 40. Typically, for dilution manifolds comprising a plurality of inner diluters such as a first inner diluter and a second inner diluter, the inner diluters are identical to each other. However, the present subject matter includes versions in which inner diluters used in a dilution manifold are different from each other such as in size, shape, and/or configuration. The inner diluter 40 defines an entryway 41, an exitway 42, and a passageway 43 extending therebetween. In many versions, the entryway 41 defines a receiving region sized and shaped to fittingly receive the outlet port 34 of the inlet cap 30, or the exitway 42 of another inner diluter 40. An O-ring or other sealing member 44 is positioned within the passageway 43 and is configured to engage and seal with an outlet port 34 of the inlet cap 30 or the exitway 42 of another inner diluter 40 inserted therein. FIG. 2C illustrates an exhaust diluter 50. The exhaust diluter 50 defines an entryway 51, and exitway 52, and a passageway 53 extending therebetween. In many versions, the entryway 51 defines a receiving region sized and shaped to fittingly receive the exitway 42 of an inner diluter 40. An O-ring or other sealing member 54 is provided within the passageway 53 and is configured to sealingly engage the exitway 42 of an inner diluter 40 inserted therein. As previously noted, the dilution manifold 20 may also include a U-turn component, which is depicted in FIG. 1C as component 58. In this version, the U-turn component 58 provides airflow communication between inner diluters 40. As will be understood, a first end of the U-turn component is sealingly engaged with the exitway 42 of one inner diluter 40, and a second end of the U-turn component is sealingly engaged with the entryway 41 of an adjacent inner diluter 40.

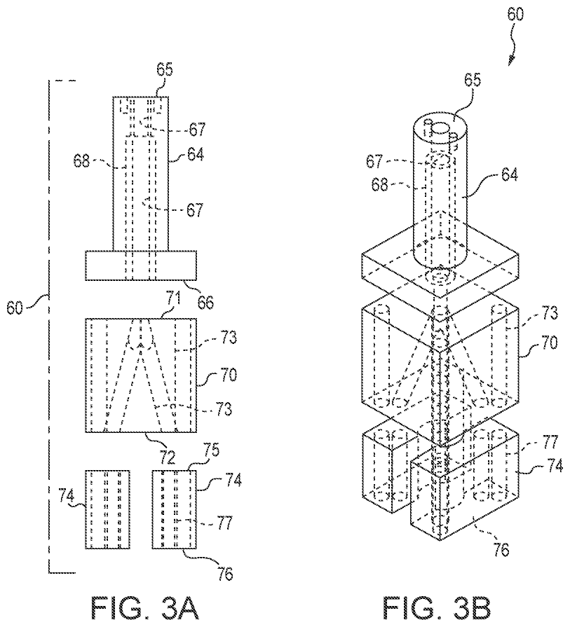

Referring to FIGS. 3A-3B, the aerosol flow splitter includes an aerosol flow adaptor which includes a static mixer sleeve, a flow splitter body, and two chamber adaptors. O-rings and alignment pins ensure an air-tight seal between components and repeatable exposure conditions. Specifically, and with further reference to FIGS. 3A and 3B, the aerosol flow splitter 60 includes an aerosol flow adapter 64, a flow splitter body 70, and at least one chamber adapter 74. The referenced figures depict first and second chamber adapters 74, respectively. The aerosol flow adapter 64 defines an upper face 65, a lower face 66, and a conduit 67 extending therebetween. In particular embodiments, the aerosol flow adapter 64 further includes a static mixer 68 which promotes mixing of fluid flow in that region. The flow splitter body 70 also defines an upper face 71, a lower face 72, and one or more conduits 73 extending therebetween. The chamber adapter 74 also defines an upper face 75, a lower face 76, and one or more conduits 77 extending between the faces 75 and 76. Upon assembly, the splitter body 70 is disposed between the aerosol flow splitter 64 and the chamber adapters 74. As will be understood, the components are sealingly engaged to each other and conduits aligned so as to provide flow communication between the upper face 65 of the aerosol flow adapter 64 and the lower face 76 of the chamber adapters 74. It will be understood that the present subject matter includes versions in which one or more components 64, 70, and 74 are formed integrally such as a one piece component of the aerosol flow adapter 64 and the flow splitter body 70.

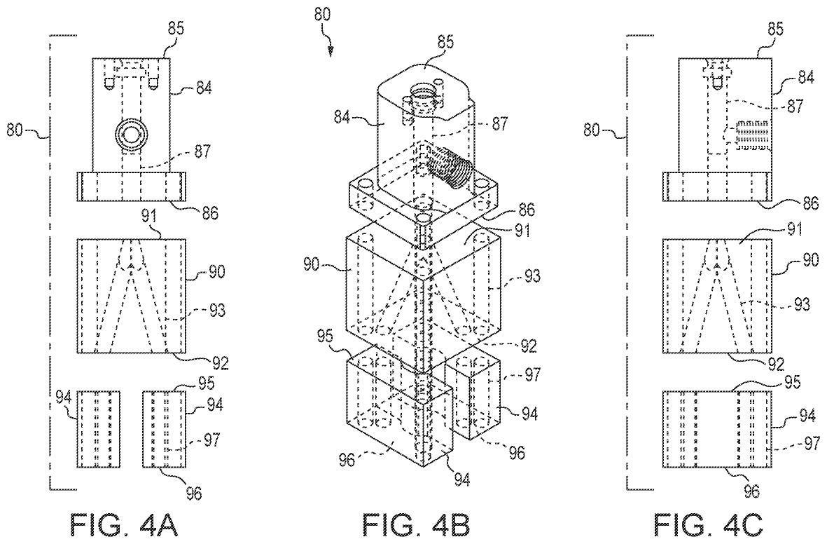

FIGS. 4A-4C illustrate a VOC flow splitter including a flow adaptor with a sampling port to allow real-time concentration analysis with GC-coupled detection methods. These figures further illustrate a sampling region. Specifically, and with further reference to FIGS. 4A-4C, the VOC flow splitter 80 includes a VOC sampling port 84, a flow splitter body 90, and at least one chamber adapter 94. The referenced figures illustrate first and second chamber adapters 94, respectively. The VOC sampling port 84 defines an upper face 85, a lower face 86, and a conduit 87 extending between the faces 85, 86. The flow splitter body 90 defines an upper face 91, a lower face 92, and one or more conduits 93 extending between the faces 91, 92. The chamber adapter 94 defines an upper face 95, a lower face 96, and one or more conduits 97 extending between the faces 95, 96. Upon assembly, the flow splitter body 90 is disposed between the VOC sampling port 84 and the chamber adapters 94. As will be appreciated, the components are sealingly engaged to each other and conduits aligned so as to provide flow communication between the upper face 85 of the VOC sampling port 84 and the lower face 96 of the chamber adapters 94. It will be understood that the present subject matter includes versions in which one or more of the components 84, 90, and 94 are formed integrally such as a one-piece component of the flow splitter body 90 and one or both of the chamber adapters 94.

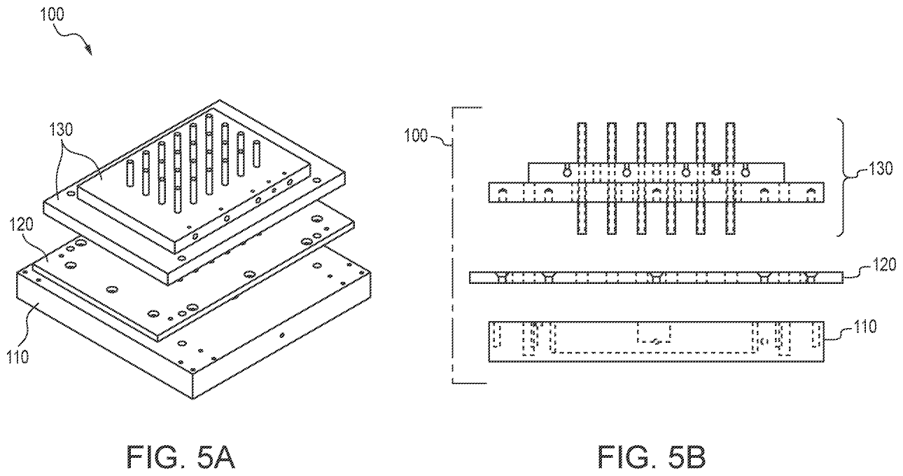

FIGS. 5A-5B are exploded views of the exposure chamber. These figures show a plenum plate with 24 stainless steel delivery nozzles, an annular plate, and a base. Specifically, the exposure chamber 100 includes a base 110, a plenum plate 130, and an annular plate 120 disposed between the base 110 and the plenum plate 130.

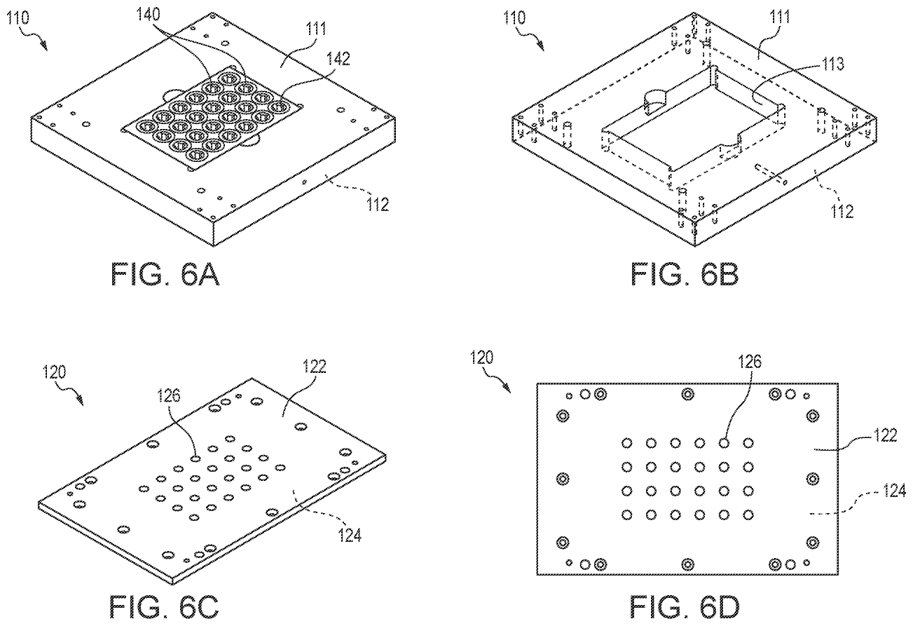

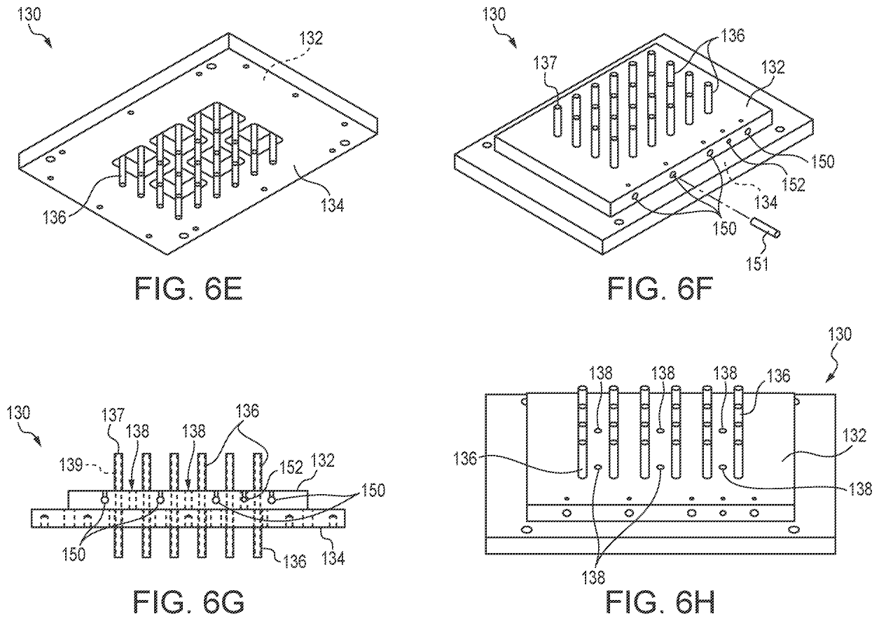

FIGS. 6A-6G illustrate components of an exposure chamber used in the cell culture exposure system. The base 110 as shown in FIGS. 6A-6B is directly compatible with a standard 24-well cell culture plate which includes 6.5 mm transwell ALI inserts. The base 110 defines an upper surface 111, and oppositely directed lower surface 112, and a culture plate receiving region 113 at least accessible from the upper surface 111. The receiving region 113 is sized and shaped to receive and support a culture plate 140 positioned therein as depicted in FIG. 6A. The culture plate 140 typically includes a plurality of wells 142, and as described herein typically 24 wells. However, it will be understood that the present subject matter includes exposure chambers and particularly bases, that are configured to receive and accommodate a wide array of culture plates such as plates having less than 24 wells, and plates having more than 24 wells. The annular plate 120 as shown in FIGS. 6C-6D, provides a direct vacuum connection to isolated quadrants of the plenum plate 130, as described in greater detail herein. The annular plate 120 defines an upper surface 122, a lower surface 124, and a plurality of apertures 126 extending between the upper and lower surfaces 122, 124. Each aperture 126 is sized and shaped to maintain an open annular space and receive a corresponding delivery nozzle of the plenum plate 130, as described herein. The plenum plate 130 is illustrated in FIGS. 6E-6H. The plenum plate 130 defines an upper surface 132, a lower surface 134, and a plurality of delivery nozzles 136 extending between and generally beyond the surfaces 132, 134. Each delivery nozzle 136 defines an upper orifice 137 and a lower orifice 138. A passage 139 extends between the orifices 137, 138 in each nozzle 136. The passages 139 provide flow communication between the upper and lower orifices 137, 138. As will be understood, the upper orifices 137 of the delivery nozzles 136 are engaged with and in flow communication with the flow splitter, i.e., either the aerosol flow splitter 60 or the VOC flow splitter 80, and receive fluid flow therefrom for directing toward a well of a cell culture plate positioned in the exposure chamber 100. A vacuum source must be connected to 138 to control flow communication between the dilution manifold and the cell exposure chamber. A significant feature of this embodiment relates to a collection of isolated quadrants which are visible along a bottom view of the plenum plate 130, see FIG. 6E. This ensures that the vacuum flow rate is balanced for the four technical replicates downstream of each dose in this embodiment, creates equivalent flow rates and pressure gradients for each well position, and eliminates crosstalk and contamination across different doses. The plenum plate 130 also allows direct installation of a plurality and typically four direct current heat cartridges and a thermocouple. In this regard, a plurality of apertures are provided and are visible in FIGS. 6F and 6G. This configuration enables a user to create a thermophoretic gradient and promote deposition of the delivered test article. Specifically and with reference to FIGS. 6F and 6G, one or more apertures 150 are defined in the plenum plate 130. The apertures 150 are sized and shaped to receive one or more heat elements 151 for example heat cartridges as known in the art. One or more additional apertures 152 are provided for receiving a thermocouple as known in the art.

In a particular version of the present subject matter exposure system, the outlet faces of the flow splitter, i.e., the lower faces 76 of the chamber adapters 74 of the aerosol flow splitter 60 or the lower faces 96 of the chamber adapters 94 of the VOC flow splitter 80, provide access to a number of outlet ends of conduits, i.e., conduits 77 or conduits 97, equal to a number of wells in a culture plate used in the exposure system. For example, if the culture plate for use in the cell exposure system provides 24 wells, and so the exposure chamber 100 includes a total of 24 delivery nozzles 136, then the total number of conduit outlets accessible along the lower face of the flow splitter, is also 24. This particular configuration is referred to herein as “an independent well configuration” thus referring to a strategy in which each well of a cell culture plate receives its own independent supply or flow of test fluid from the flow splitter. As a consequence of this particular configuration, no mixing of test fluid occurs downstream of the flow splitter and upstream of the well(s).

The present subject matter also provides various methods, and particularly methods for exposing a cell culture plate to a test fluid flow. In a particular embodiment, such method comprises providing a cell culture exposure system including at least one dilution manifold including at least one inlet for receiving a fluid flow, at least one flow splitter, and an exposure chamber configured to receive a cell culture plate. The method also comprises providing a cell culture plate having at least one well. The method additionally comprises positioning cell culture material in the at least one well. The method further comprises placing the cell culture plate with cell culture material positioned therein, in the exposure chamber of the cell culture exposure system. Then, the method comprises directing a fluid flow into the at least one inlet of the at least one dilution manifold such that the fluid flow passes through the at least one well and the cell culture material positioned therein. In a specific version of the method, the cell culture plate includes 24 wells. And, in this method, a user may wish to utilize particular parameters such as the fluid flow exhibiting a temperature of 37° C. and a relative humidity greater than 80%.

A modular flow splitter configuration creates four technical replicates with uniform deposition for VOCs and fine particles, for example less than 2.5 μm. Alternate embodiments of the flow splitter component include updates to allow real-time sampling of VOCs.

Alternative embodiments include a plenum plate configured to fittingly engage other nozzle assemblies besides a conventional 24 stainless steel nozzle assembly. The plenum plates can be adapted to optimize delivery of inhaled test articles such as but not limited to: (i) altered inner diameter or outer diameter, (ii) altered height of nozzles to optimize delivery parameters, and (iii) shape of nozzle outlet above cells.

Alternative embodiments of the dilution manifold include the use of a plurality and typically two split manifolds, for example each with three dilutors, one full manifold, for example with six dilutors with a U-turn connector that directly attaches to the flow splitters. Alternatively, six dilutors can be connected in series with an external heat source (not shown) to aid transport of reactive gases and volatile compounds that require high heat to remain volatile.

Alternative embodiments of the system of the present subject matter include adapted designs for 6- and 12-well plates which achieve serial dilution. Such system may include one or more of the following modifications: shortened dilution manifolds to provide 3-4 doses per plate, updated flow splitters which feature a two-way split with an integrated static mixer sleeve or sampling port, and an updated plenum plate with a reduced number of nozzles centered over 6- or 12-well inserts.

Two technical replicates per dose in the exposure chamber can be achieved by an isolated plenum space over two wells which balances the vacuum flow rates experienced by each well.

Contrary to prior art systems, the present subject matter provides a cell culture exposure system that are flexible and compatible with fine particles (<2.5 μm) as well as VOCs, reactive gases, and ultrafine particles (<0.1 μm) under dynamic airflow conditions. The modular dilution manifold, flow splitters, and sealed exposure chamber accomplish serial dilution and produce six doses with four technical replicates per dose within a standard 24-well cell culture plate while maintaining appropriate cell culture conditions (T=37° C., RH>80%). Notably, standard deviation of technical replicates of particle deposition is often less than 10% with liquid fluorescein particles with a median mass aerodynamic diameter (MMAD) of 1.5 μm. The geometry of this system is optimized for low flow rates which minimize cell cytotoxicity due to shear stress.

In a particular embodiment, a system of the present subject matter provides serial dilution of VOC and aerosols to achieve six (6) doses with four (4) technical replicates per dose.

Although the system described in US Patent Publication No. 2018/0171280 claims the same achievement, the plenum design fails to achieve balanced flows so four technical replicates cannot be achieved downstream of each dose for VOCs. Moreover, that system cannot transport aerosols and fails to achieve serial dilution of particles.

In another particular embodiment, a system of the present subject matter utilizes standard 24-well cell culture plates with 6.5 mm inserts to increase throughput and minimize sampling handling time. Therefore, inserts do not need to be individually transferred from cell culture plate to the exposure apparatus.

As far as is known, the system described in US Patent Publication No. 2018/0171280 is also compatible with standard 24-well plates and achieves serial dilution under dynamic airflow conditions, but the geometry of the exposure unit is incompatible with particles. The shared plenum space creates undesirable pressure gradients and variable airflow rates which prevents repeatable particle deposition or VOC delivery within technical replicates.

Many other benefits will no doubt become apparent from future application and development of this technology.

All patents, applications, standards, and articles noted herein are hereby incorporated by reference in their entirety.

The present subject matter includes all operable combinations of features and aspects described herein. Thus, for example if one feature is described in association with an embodiment and another feature is described in association with another embodiment, it will be understood that the present subject matter includes embodiments having a combination of these features.

As described hereinabove, the present subject matter solves many problems associated with previous strategies, systems and/or devices. However, it will be appreciated that various changes in the details, materials and arrangements of components, which have been herein described and illustrated in order to explain the nature of the present subject matter, may be made by those skilled in the art without departing from the principle and scope of the claimed subject matter, as expressed in the appended claims.

What is claimed is:

1. A cell culture exposure system comprising:
at least one dilution manifold including at least one inlet for receiving a fluid flow;
at least one flow splitter;
an exposure chamber configured to receive a cell culture plate;
wherein the at least one flow splitter is disposed between the at least one dilution manifold and the exposure chamber, and provides flow communication between the at least one dilution manifold and a cell culture plate received in the exposure chamber.

2. The cell culture exposure system of claim 1 wherein the at least one dilution manifold includes:
at least one inlet cap having the at least one inlet;
at least one inner diluter;
at least one exhaust dilutor.

3. The cell culture exposure system of claim 2 wherein the at least one dilution manifold includes at least two inner diluters and the at least one dilution manifold further includes:
a U-turn component disposed between the at least two inner diluters and providing flow communication therebetween.

4. The cell culture exposure system of claim 1 wherein the flow splitter is a VOC flow splitter.

5. The cell culture exposure system of claim 4 wherein the VOC flow splitter includes:
a sampling port;
a flow splitter body;
at least one chamber adapter.

6. The cell culture exposure system of claim 1 wherein the flow splitter is an aerosol flow splitter.

7. The cell culture exposure system of claim 6 wherein the aerosol flow splitter includes:
an aerosol flow adapter;
a flow splitter body;
at least one chamber adapter.

8. The cell culture exposure system of claim 7 wherein the aerosol flow adapter further includes a static mixer.

9. The cell culture exposure system of claim 1 wherein the exposure chamber includes:
a plenum plate;
an annular plate;
a base configured to receive the cell culture plate;
wherein the annular plate is disposed between the plenum plate and the base.

10. The cell culture exposure system of claim 1 wherein the exposure chamber further includes a heating element.

11. The cell culture exposure system of claim 1 further comprising:
a cell culture plate disposed in the exposure chamber.

12. The cell culture exposure system of claim 1 wherein cell culture plate includes a plurality of wells, and the system is configured such that each well of the plurality of wells receives its own independent flow of the fluid flow from the at least one inlet.

13. A cell culture exposure system comprising:
a dilution manifold having at least one inlet for receiving a fluid flow;
an assembly of flow splitters;
an exposure chamber configured to receive a cell culture plate having a plurality of wells;
wherein the assembly of flow splitters is disposed between the dilution manifold and the exposure chamber and provides flow communication therebetween and the system is configured such that each well of the plurality of wells receives its own independent flow of the fluid flow downstream of the assembly of flow splitters.

14. The cell culture exposure system of claim 13 wherein the dilution manifold includes:
at least one inner diluter;
at least one exhaust diluter.

15. The cell culture exposure system of claim 13 wherein the assembly of flow splitters is selected from a VOC flow splitter and an aerosol flow splitter.

16. The cell culture exposure system of claim 13 wherein the exposure chamber includes:
a plenum plate;
an annular plate;
a base configured to receive the cell culture plate;
wherein the annular plate is disposed between the plenum plate and the base.

17. The cell culture exposure system of claim 13 further comprising:
a cell culture plate disposed in the exposure chamber.

18. A method for exposing a cell culture plate to a test fluid flow, the method comprising:
providing a cell culture exposure system including at least one dilution manifold including at least one inlet for receiving a fluid flow, at least one flow splitter, and an exposure chamber configured to receive a cell culture plate;
providing a cell culture plate having at least one well;
positioning cell culture material in the at least one well;
placing the cell culture plate with cell culture material positioned therein, in the exposure chamber of the cell culture exposure system;
directing a fluid flow into the at least one inlet of the at least one dilution manifold such that the fluid flow passes through the at least one well and the cell culture material positioned therein.

19. The method of claim 18 wherein the cell culture plate includes 24 wells.

20. The method of claim 18 wherein the fluid flow exhibits a temperature of 37° C. and a relative humidity greater than 80%.
